(19) United States
(12) Patent Application Publication
(10) Pub. No.: US 2026/0041826 A1
(43) Pub. Date: Feb. 12, 2026

(54) DEVICE FOR DIALYSIS TREATMENT

(71) Applicant: FRESENIUS MEDICAL CARE DEUTSCHLAND GMBH, Bad Homburg (DE)

(72) Inventors: Frank HEDMANN, Bad Homburg (DE); Torsten HOCHREIN, Bad Homburg (DE)

(73) Assignee: FRESENIUS MEDICAL CARE DEUTSCHLAND GMBH, Bad Homburg (DE)

(21) Appl. No.: 19/123,073

(22) PCT Filed: Oct. 23, 2023
(86) PCT No.: PCT/EP2023/079468

§ 371 Date: Apr. 22, 2025

(30) Foreign Application Priority Data
Oct. 27, 2022 (DE) 10 2022 128 529.4

(51) Int. Cl. A61M 1/14; A61M 1/28

(52) U.S. Cl. CPC A61M 1/159; A61M 1/153; A61M 1/155; A61M 1/1565; A61M 1/281; A61M 1/282; A61M 2205/3331

(57) ABSTRACT

The present invention relates to a device for dialysis treatment, in particular a device for peritoneal dialysis, having first and second discontinuous pumps that are switchable by means of at least two valves and having a control for generating a continuous volume flow of dialyzate, characterized in that the control is adapted to carry out at least one of the following steps: a) suspending at least one pressure measurement during a switching time of at least one of the valves; b) calculatory compensation of a portion of a result of a pressure measurement based on unit properties during a switching time of at least one of the valves; c) postponing a valve switching point in time of the second pump so that the valve switching point in time of the second pump does not coincide with a valve switching point in time of the first pump; and d) adaptive postponement of a valve switching point in time of the second pump so that said valve switching point in time does not coincide with a valve switching point in time of the first pump, wherein use is preferably made by the control of an optimization algorithm for determining a point in time to which the valve switching point in time of the second pump is postponed.

The present invention relates to a device for dialysis treatment, in particular to a device for peritoneal dialysis, that is configured to provide a continuous volume flow of dialyzate by means of two discontinuously operating pumps. The present invention furthermore relates to a corresponding method.

It is essential in the continuous conveying of dialyzate by means of a device for extracorporeal blood treatment, in particular a device for peritoneal dialysis, by means of which dialyzate is conveyed directly into and out of the abdominal cavity of a patient that the negative pressures or excess pressures specified for the conveying are always correctly observed since deviations therefrom are frequently accompanied by indisposition on the part of a patient and may even cause injury to the peritoneum. The conveying of dialyzate into and out of a patient should in particular takes place as mildly as possible in treatments in pediatrics and should therefore take place at a smaller negative pressure than in adult therapy.

The transfer times of the dialyzate to be conveyed should furthermore be as short as possible so that the prescribed times for the inflow phase, the dwell time, and the outflow time are observed as exactly as possible. It is beneficial here for an active pump to always be operated at the highest possible flow rate.

Disruptions to the pumping procedure of dialyzate into and out of the patient should furthermore generally be avoided.

It has been found in practice that a constant provision of a continuous volume flow of dialyzate corresponding to the prescription is in particular subject to problems in the outflow of peritoneal dialysis treatment.

High demands are in particular made in the outflow phase of a peritoneal dialysis treatment since the required vacuums are, for example, at a level of −100 mbar and they—as well as any unwanted deviations therefrom—can be noticeably perceived by the patient. In pediatric peritoneal dialysis treatments, the specified pressures are additionally further reduced to a minimum of −80 mbar, which further increases the demands.

Against this background, it is the underlying object of the present invention to alleviate or even fully eliminate the problems known from the prior art. It is in particular the underlying object of the present invention to provide a device and a method by means of which a continuous volume flow of dialyzate can also be achieved with said high demands.

The object is achieved by a device having the features of claim 1 and by a method having the features of claim 10. Advantageous further developments of the invention are the subject of the dependent claims.

A device for extracorporeal blood treatment, in particular a device for peritoneal dialysis, is accordingly provided having first and second discontinuous pumps that are connectable by means of at least two valves and having a control to generate a continuous volume flow of dialyzate.

In accordance with the invention, the control is adapted to carry out at least one of the following steps: a) suspending at least one pressure measurement during a switching time of at least one of the valves; b) calculatory compensation of a portion of a pressure measurement based on unit properties during a switching time of at least one of the valves; c) postponing a valve switching point in time of the second pump so that the valve switching point in time of the second pump does not coincide with a valve switching point in time of the first pump; and d) adaptive postponing of a valve switching point in time of the second pump so that said valve switching point in time does not coincide with a valve switching point in time of the first pump, wherein use is preferably made by the control of an optimization algorithm for determining a point in time to which the valve switching point in time of the second pump is postponed.

Pump cassettes are often used in practice that have two pump chambers that are associated with respective first and second pumps and that are fluidically connected to one another via at least one valve.

If, for example, the first pump is active and conveys dialyzate, the second pump is conventionally switched in while the first pump is still active. A valve fluidically connecting the first pump to the second pump is opened for this purpose. As soon as the valve is opened, however, a pressure compensation takes place between the first and second pumps or their pump chambers. This has the result that a measured conveying pressure of the first pump is falsified, whereupon the active first pump is stopped.

The continuous volume flow of dialyzate to be achieved by the concerted action of the first and second pumps is interrupted in an unwanted manner due to the falsification of the conveying pressure of the first pump by the switching in of the second pump.

The occurrence of such a falsification of the conveying pressure can preferably be suppressed by the present invention and/or a measured change of the conveying pressure can be recognized as a falsification of the conveying pressure by a switching in of the second pump and/or can thereupon be compensated or left out of consideration.

The influence of a falsification of the conveying pressure of the first pump by the switching in of the second pump in response to a measurement of a conveying pressure (for example by a suspension of the pressure measurement during the switching in or by a calculatory compensation of a pressure change taking place due to the switching in) and/or in response to an operation of the first and/or second pump(s) (for example an unwanted switching off of the first pump due to the falsification of a measured conveying pressure of the first pump) is preferably avoided as much as possible.

A device in accordance with the invention thus preferably makes possible an avoidance of disrupting pressure pulses due to the switching of at least one valve that is associated with the first or second pump.

The present invention is here not restricted to a specific conveying system or pump system, but can rather be used in any embodiment using two discontinuous pumps in which a continuous volume flow of dialyzate should be achieved.

For example, with a device in accordance with the invention, an active pressure measurement can take place in a measurement path that could be disrupted by valve or patient clamp switches. A case is thus covered by the invention in which the conveying pressure of the first pump and/or of the second pump takes place in a measurement path.

In other words, the present invention can represent a possibility of avoidance and/or compensation of pressure fluctuations (e.g. pressure pulses) in a device for dialysis treatment, with a focus of the invention preferably being on the inclusion of the components (valves) acting on fluid and their switching. Actuators of the flow passages such as valves, clamps, or any other elements suitable for regulating the fluid flow are thus preferably acted on in accordance with the invention and not the conveying system itself (e.g. pump).

The method steps that a control of a device in accordance with the invention can carry out will be described in more detail in the following.

In accordance with an embodiment of the invention, the control is adapted to carry out at least the following step: a) suspending at least one pressure measurement during a switching time of at least one of the valves.

In other words, a measurement of the conveying pressure of the first pump is preferably interrupted during a valve switching period of the first and/or second pump. Falsifications or artifacts of a measured conveying pressure arising due to the valve switching do not enter into the measurement of the conveying pressure in this manner.

The measurement of the conveying pressure is preferably suspended up to a detected system calming.

An advantage of this procedure comprises this step being simple to implement.

In accordance with an embodiment of the invention, the control is adapted to carry out at least the following step: b) calculatory compensation of a portion of a result of a pressure measurement based on unit properties during a switching time of at least one of the valves by calculation.

In this embodiment, the control preferably detects a switching of at least one valve fluidically connecting the first and second pumps, for example to couple or switch in the second pump, being imminent and compensates the effect of the switching on a measurement of a conveying pressure of the first pump, preferably while accessing a stored or taught characteristic or a stored or taught pressure profile.

The first or second pump can be acted on by different speeds for the teaching process, for example at the end of a setup process on the filling of the cassette. The relevant valve paths are established during this movement and the pressure fluctuations that arise here are recorded. The time, the switching speed, and the pressure pulse can be determined from this record. These parameters can be used in the further course of the treatment.

An advantage of this procedure comprises the pressure measurement not having to be interrupted, but rather the conveying pressure of the pump(s) being able to be continuously measured.

This advantage can also be achieved if, in accordance with an embodiment of the invention, the control is adapted to carry out at least the following step: c) postponing a valve switching point in time of the second pump so that the valve switching point in time of the second pump does not coincide with a valve switching point in time of the first pump. In other words, the valve switching point in time should preferably deviate in time from a valve switching point in time of the first pump.

A control preferably takes place such that the first pump end its pump stroke and the second pump is switched in with a delay after the pump stroke.

The switching in of the second period can take place, for example, after the elapse of a fixed delay period, for example of some milliseconds.

In accordance with an embodiment of the invention, the control is adapted to carry out at least the following step: d) adaptively postponing a valve switching point in time of the second pump so that this valve switching point in time does not coincide with a valve switching point in time of the first pump, with use preferably being made on the part of the control of an optimization algorithm to determine a point in time to which the valve switching point in time of the second pump is postponed.

The term “adaptively” postponing is preferably to be understood here such that a time period by which a valve switching point in time of the second pump is delayed or postponed relative to a valve switching point in time of the first pump is not fixed or constant, but is rather determined individually in each case for the circumstances obtaining at a current point in time. The duration of the determination of the time period is thus preferably individually matched or adapted to the circumstances obtaining at a current point in time.

At least one of the following parameters is preferably taken into account by the optimization algorithm on the determination of the duration of the time period:
flow rate (pump speed) of the preferably active pump
degree of filling of the patient (dialyzate volume in the patient)
a pump chamber volume last conveyed by the preferably active pump
treatment type (therapy of adults or pediatrics)

A suspension of the pressure measurement can likewise be avoided by means of this embodiment and measured values of the conveying pressure of the first and/or second pump(s) can be continuously recorded.

In addition, a maximum flow rate (pump speed) of the first and/or second pump(s) can preferably be achieved.

The measurable flow displacements and pressure pulses, in other words the unwanted falsifications of the measured conveying pressure, can also be reliably eliminated at relatively small flow rates (pump speeds).

The following advantages can be achieved overall by means of the present invention: Improved provision of a continuous volume flow of dialyzate, in particular in the outflow phase of a peritoneal dialysis treatment; reduced error messages due to falsified pressure measurements; improved adaptation of the pump control to the individual patient outflow behavior; and higher performance of dialysis units in pediatric therapy.

In accordance with an embodiment of the invention, the control is adapted to carry out at least one of the steps in an inflow phase of a peritoneal dialysis treatment and/or in an outflow phase of a peritoneal dialysis treatment.

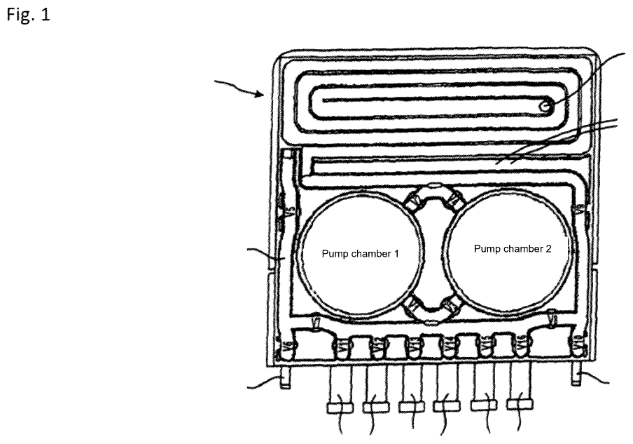

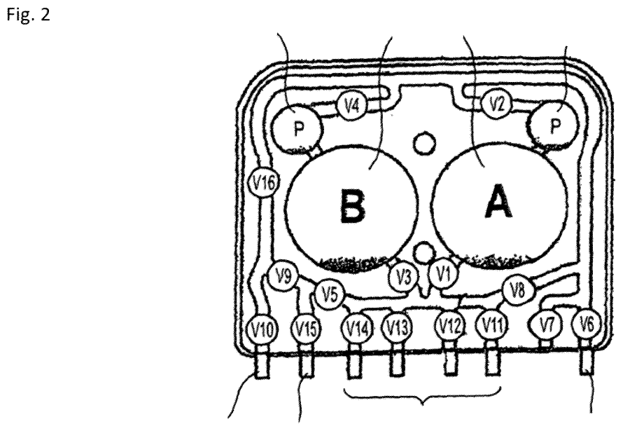

In accordance with an embodiment, the control is adapted only to open a valve fluidically connecting the first pump to the second pump when the second pump has completely ended a last pump stroke and/or is inactive. It is thus prevented that a pressure compensation generated by the valve opening falsifies measurements of the conveying pressure of the first pump. Examples of such a valve fluidically connecting the first pump to the second pump are, for example, the valves V1 and V3 in FIGS. 1 and 2.

In accordance with an embodiment, the first and second pumps each interact with a pump chamber formed in a disposable article (or simply disposable) to convey fluid and the at least two valves are preferably each components of the disposable.

In accordance with an embodiment, use is made of a pressure profile from a database associated with the unit properties, in particular with a valve switching, as part of the calculatory compensation in order thus to identify and compensate the portion of the result of a pressure measurement based on the unit properties during a switching time of at least one of the valves.

The pressure profile can be prepared by means of a teaching process described above.

In accordance with an embodiment, the control is adapted to make use of the optimization algorithm to determine a point in time or to calculate a point in time in advance to which the valve switching point in time of the second pump should be postponed or is postponed, with the optimization algorithm taking account of at least one of the following parameters at a given point in time: Flow rate of the first and/or of the second pump, preferably flow rate of the pump of the first and second pumps that is active at the point in time; last conveyed pump chamber volume of the first and/or second pump(s), preferably the flow rate of the pump of the first and second pumps that is active at the point in time; volume present in the patient and type of a treatment carried out.

After the advance calculation of the desired valve switching point in time of the second pump by means of the control or of the optimization algorithm, the control preferably controls the second pump such that its valve switching point in time falls on the point in time calculated in advance.

In accordance with an embodiment, the optimization algorithm is adapted to minimize a time delay between a valve switching point in time of the second pump to start a pump stroke of the second pump and a valve switching point in time of the first pump on the ending of a pump stroke of the first pump.

Step c) can include a postponement of the valve switching point in time of the second pump by a fixed delay period starting from a valve switching point in time of the first pump such that the valve switching point in time of the second pump does not coincide with the valve switching point in time of the first pump but rather deviates therefrom in time. The fixed delay period can be stored in a database, for example.

Step d) can furthermore include a postponement of the valve switching point in time of the second pump by a variable delay period, preferably individually determined by the optimization algorithm for a specific valve switching procedure or for every valve switching procedure starting from a valve switching point in time of the first pump such that the valve switching point in time of the second pump does not coincide with the valve switching point in time of the first pump.

Another aspect of the invention relates to a method of generating a continuous volume flow of dialyzate by means of a device for dialysis treatment, preferably a device for dialysis treatment in accordance with the present invention, having first and second discontinuous pumps that are switchable by means of at least two valves, wherein the method comprises at least one of the following steps: a) suspending at least one pressure measurement during a switching time of at least one of the valves; b) calculatory compensation of a portion of a result of a pressure measurement based on unit properties during a switching time of at least one of the valves; c) postponing a valve switching point in time of the second pump so that the valve switching point in time of the second pump does not coincide with a valve switching point in time of the first pump; and d) adaptive postponing of a valve switching point in time of the second pump so that said valve switching point in time does not coincide with a valve switching point in time of the first pump, wherein use is preferably made of an optimization algorithm for determining a point in time to which the valve switching point in time of the second pump is postponed.

All the features disclosed above in the context of a device in accordance with the invention are equally applicable to a method in accordance with the invention even if they are not explicitly presented again, to avoid redundancies and vice versa.

In a method in accordance with the invention, at least one of the steps can be carried out in an inflow phase of a peritoneal dialysis treatment and/or in an outflow phase of a peritoneal dialysis treatment.

In accordance with an embodiment, a method in accordance with the invention provides that a valve fluidically connecting the first pump to the second pump is only opened when the second pump has completely ended a last pump stroke and/or is inactive and/or a measurement of the conveying pressure of the first pump has ended.

In accordance with an embodiment, a method in accordance with the invention provides that use is made of a pressure profile from a database associated with the unit properties, in particular with a valve switching, as part of the calculatory compensation in order thus to identify and compensate the portion of the result of a pressure measurement based on the unit properties during a switching time of at least one of the valves. The calculatory compensation can, for example, comprise a subtraction of the pressure profile accompanying a valve switching from a measured pressure profile.

In accordance with an embodiment, in a method in accordance with the invention, use is made of the optimization algorithm to determine a point in time or to calculate a point in time in advance to which the valve switching point in time of the second pump is postponed, with the optimization algorithm taking account of at least one of the following parameters at a given point in time: Flow rate of the first and/or of the second pump, preferably flow rate of the pump of the first and second pumps that is active at the point in time; last conveyed pump chamber volume of the first and/or second pump(s), preferably the flow rate of the pump of the first and second pumps that is active at the point in time; volume present in the patient and type of a treatment carried out.

The optimization algorithm can adapted to minimize a time delay between a valve switching point in time of the second pump to start a pump stroke of the second pump and a valve switching point in time of the first pump on the ending of a pump stroke of the first pump.

In accordance with an embodiment of a method in accordance with the invention, step c) includes a postponement of the valve switching point in time of the second pump by a fixed delay period starting from a valve switching point in time of the first pump such that the valve switching point in time of the second pump does not coincide with the valve switching point in time of the first pump or deviates therefrom in time.

In accordance with an embodiment of a method in accordance with the invention, step d) includes a postponement of the valve switching point in time of the second pump by a variable delay period, preferably individually determined by the optimization algorithm for every valve switching procedure, starting from a valve switching point in time of the first point such that the valve switching point in time of the second pump does not coincide with the valve switching point in time of the first pump.

It must be pointed out at this point that the present disclosure is to be understood such that features that are disclosed as part of a specific feature combination or embodiment can also be claimed in isolation or in other feature combinations and the disclosure is in no case restricted to the explicitly named feature combinations. All the feature combinations covered by the present disclosure are only not explicitly disclosed for reasons of brief and concise wording.

It must furthermore be pointed out that if an element is named in the singular with the article “a” or “one”, this is not to be interpreted as “exactly one”, but that rather an embodiment having the relevant element in the plural is also covered by the disclosure and vice versa.

Further advantages, effects and features of the present invention result from the following description of embodiments of the invention while referring to the Figures in which reference numerals that are the same designate the same or similar components. There are shown:

FIG. 1 a pump cassette configured as a disposable in accordance with a first embodiment that can be used within the framework of the present invention;

FIG. 2 a pump cassette configured as a disposable in accordance with a second embodiment that can be used within the framework of the present invention;

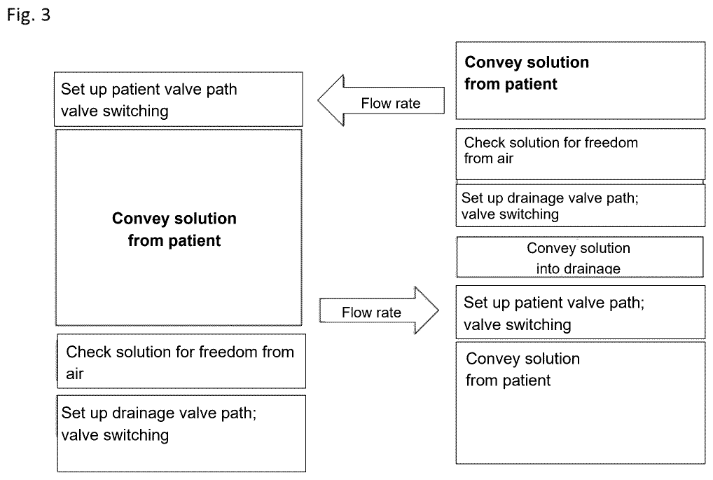

FIG. 3 a flowchart that shows the method steps respectively carried out by a first pump and a second pump as part of an embodiment of the present invention; and

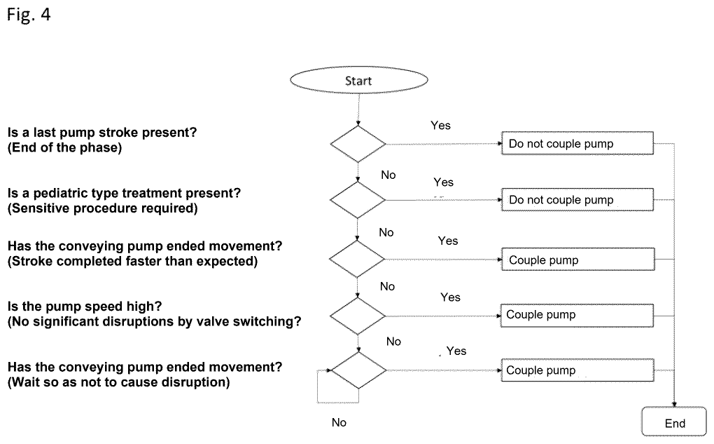

FIG. 4 an example of an optimization algorithm that is used as part of an embodiment of the present invention.

A first embodiment of a cassette is shown in FIG. 1. It has a hard part 1 of plastic in which the fluid paths and coupling regions are introduced as corresponding cutouts, chambers, and channels. The hard part can be produced here e.g. as an injection molded part or as a deep drawn part. The coupling plane of the hard part 1 is covered by a flexible film 2 that is welded to the hard part in a marginal region. The flexible film 2 is pressed together with the hard part by the pressing of the cassette to a coupling surface of the dialysis machine. The fluid paths within the cassette are separated from one another in a fluid tight manner by the pressing of the flexible film to the web regions of the hard part.

The cassette has connections to connect the cassette to the other fluid paths. On the one hand, a connection 3 is provided for connection to a drain and a connection 4 is provided for connection to the connector. Corresponding hose elements that are not shown in FIG. 1 can be provided at these connections. The cassette furthermore has a plurality of connectors 5 to connect dialyzate containers. The connectors 5 are here designed as connectors, for example, to which corresponding connector elements can be connected,

The connections are each connected to fluid paths within the cassette. Valve regions are provided in these fluid paths. The flexible film 2 can be pressed via valve actuators at the machine side into the hard part 1 in these valve regions so that the corresponding fluid path is blocked. The cassette here first has a corresponding valve for each connection via which this connection can be opened or closed. The valve V10 is here associated with the connection 3 for the drainage and the valve V6 is associated with the connection 4 for the patient connector. Valves V11 to V16 are associated with the connections 5 for the dialyzate container 10.

Pump chambers 6 and 6′ that can be actuated by corresponding pump actuators of the dialysis machine are furthermore provided in the cassette. The pump chambers 6 and 6′ are here concave cutouts in the hard part 1 that are covered by the flexible film 2. The film can now be pressed into the pump chambers 6 and 6′ and can be pulled out of these pump chambers again by pump actuators of the dialysis machine. A pump flow through the cassette can herby be generated in conjunction with the valves V1 to V4 that switch the inlets and outflows of the pump chambers 6 and 6′. The pump chambers here are connectable to all the connections of the cassette via corresponding valve switchings.

The pump chambers 6 and 6′ can be fluidically coupled with one another via the valves V1 to V4, which can lead to a pressure compensation in an active pump chamber and thus to the initially described problems of falsification of a measured value of the conveying pressure.

A heating region 7 is furthermore integrated in the cassette in this example. The cassette is brought into contact with heating elements of the dialysis machine in this region that heat the dialyzate flowing through this region of the cassette. The heating region 7 here has a channel for the dialyzate that extends in spiral form over the heating region 7. The channel here is formed by webs of the hard part that are covered by the flexible film 2. The heating region can be provided at both sides of the cassette or can also only be provided on one side of the cassette.

Embodiments of the cassette are furthermore possible in which a heating element is integrated in the cassette. An electrical heating element such as a heating coil can here be cast into the hard part of the cassette. A heating element on the machine side can hereby be dispensed with and the flow heating can be integrated in the cassette. Electrical contacts for connecting the electrical heating element are arranged at the cassette here. The cassette furthermore has sensor regions 8 and 9 by which e.g. temperature sensors of the dialysis machine can be coupled to the cassette.

The second embodiment of a cassette shown in FIG. 2 again has fluid paths that can be opened and closed via valve regions that are here likewise consecutively numbered from V1 to V16. The cassette furthermore has connections for connection to further components of the fluid system. The connection 3 is here again provided for connection to the drain and connection 4 is provided for connection to the connector to the patient. Connections 5 are furthermore provided for the connection of dialysis containers. Each of the pump chambers 6 and 6′ has a pressure sensor 10 associated with it by means of which a conveying pressure of the associated pump chamber can be measured.

Unlike the cassette of FIG. 1, the cassette shown in FIG. 2 has a further connection 11 to connect a heating pouch. The liquid here can be pumped into a heating pouch via the connector 11 to heat the liquid from the dialyzate containers. This heating pouch lies on a heating element so that the liquid present in the heating pouch can be heated. The liquid is thereupon pumped from the heating pouch to the patient.

To achieve a continuous volume flow of dialyzate, dialyzate is alternatingly conveyed by means of the pump chambers 6 and 6′. While, for example, the pump chamber 6 removes (sucks) fluid from the patient, the pump chamber 6′ conveys the balanced volume into the drainage and is then coupled to the connection line to a patient again in a conventional manner so that the second pump can continue the next suction stroke without any time delay. This process is repeated until the prescribed treatment volume has been removed from the patient.

As described above, this direct repeat coupling of the emptied pump chamber 6′ results in measurable liquid displacements and pressure pulses that can falsify measured results of the pressure sensors 10. Volume displacements, that are respectively detectable by the pressure sensors 10, occur in the interior of the cassette due to the movements of the pump actuators of the dialysis machine that press the film 2 into the pump chambers 6 and 6′ or out of these pump chambers again. These movements of the pump actuators of the dialysis machine can thus bring about disruptive pump effects that can falsify a pressure measurement by means of the pressure sensors 10 in addition to physical effects.

Friction, pump play, and breakaway torques can be considered as examples for pump effects. The mass inertia of the dialysis solution, the flow resistance of the hose, or a potential patient line taper can be considered as examples for physical effects. All or at least a number of these disruptive effects can be bypassed or reduced by means of the present invention.

FIG. 3 illustrates the method steps respectively carried out by a first pump (left side) and a second pump (right side) as part of an embodiment of the present invention. A time axis runs from top to bottom in FIG. 3. The delay periods between the valve switching points in time of the first pump and the valve switching points in time of the second pump are drawn as Dt in FIG. 3. It thus already becomes clear at a first glance that the valve switching points in time of the first and second pumps deviate from one another in time.

In a first step S1, a valve switching takes place in the first pump to connect the pump to a patient port. The first pump thereupon conveys solution from the patient in step 2. After the conveying of solution from the patient by the first pump in step 2, the solution is checked for freedom from air in step S3. The valves of the first pump are thereupon switched over in step S4 such that the first pump is connected to a drainage or to a drain and conveys solution into the drain in a following step.

The second pump first conveys solution from the patient in step S5. A check is thereupon made in step S6 whether the solution is free of air. The valves of the second pump are switched over in step S7 such that the second pump is connected to the drainage or to the drain and conveys solution into the drain in the following step S8.

As can be recognized in FIG. 3, the valve switching points in time of the first pump in steps S1 and of the second pump in step S7 are offset in time and therefore deviate from one another. The valve switching point in time of the second pump in step S7 is thus postponed by a delay period Dt relative to the valve switching point in time of the first pump in step S1.

In step S9, a valve switching takes place in the second pump to connect the pump to a patient port. The second pump thereupon convey solution from the patient in step S10. The valve switching points in time of the first pump in step S4 and of the second pump in step S9 are also offset in time, as shown in FIG. 3, and therefore deviate in time from one another.

FIG. 4 illustrates an example of an optimization algorithm with reference to a decision tree. Such an optimization algorithm can be carried out, for example, by a control of a device in accordance with the invention. A logical determination whether the second pump should be coupled in at this point in time can be made with reference to the shown decision algorithm for a specific point in time. The algorithm first starts at Start.

A check is then made at hash #1 whether a last pump stroke is present. Such a last pump stroke can be present, for example, at the end of a phase, for example an outflow phase, when there is no longer any dialyzate volume in the patient. If a last pump stroke is present, the second pump is not coupled in (for example because no further dialyzate should be conveyed in the outflow phase) and the algorithm is ended. If no last pump stroke is present, the optimization algorithm proceeds to hash #2.

A check is made at hash #2 whether a treatment of a specific kind is present, for example a pediatric treatment. If a pediatric treatment is present, for example, the second pump is not coupled and the algorithm is ended. If no pediatric treatment is present, the optimization algorithm proceeds to hash #3.

A check is made at hash #3 whether a conveying pump, for example the first pump, has ended its movement. If the check shows that the pump has ended its movement, the second pump can be coupled in. The check in accordance with hash #3 has the advantage that, if a stroke of the conveying first pump has ended faster than expected, the second pump can be coupled in directly as soon as the movement of the first pump has ended. Any unnecessary time delay is thus avoided and the switching in point in time is individually adapted to the circumstances. If the check at hash #3 shows that the conveying pump has not yet ended its movement, the optimization algorithm proceeds to hash #4.

A check is made at hash #4 whether the pump speed of the conveying pump is high or is above a specific limit value from which onward a valve switching only influences the pressure measurement by a negligible portion. Such a limit value can be 100 ml/mi, for example. If the pump speed of the conveying pump is above the specific limit value, the second pump is coupled in. If the pump speed of the conveying pump is below the specific limit value, the optimization algorithm proceeds to hash #5.

A check is made at hash #5 whether a conveying pump, for example the first pump, has ended its movement. If the check shows that the pump has ended its movement, the second pump can be coupled in. The switching in of the second pump is thus delayed in this step until it can be expected that the switching in of the second pump longer has any disruptive effect on the operation of the first pump.

If the check at hash #5 shows that the pump has not yet ended its movement, the check at hash #5 is repeated for so long until the pump has ended its movement.

1. A device for dialysis treatment having first and second discontinuous pumps that are switchable by means of at least two valves and having a control for generating a continuous volume flow of dialyzate, wherein the control is adapted to carry out at least one of the following steps: a) suspending at least one pressure measurement during a switching time of at least one of the valves; b) calculatory compensation of a portion of a result of a pressure measurement based on unit properties during a switching time of at least one of the valves; c) postponing a valve switching point in time of the second pump so that the valve switching point in time of the second pump does not coincide with a valve switching point in time of the first pump; and d) adaptive postponement of a valve switching point in time of the second pump so that said valve switching point in time does not coincide with a valve switching point in time of the first pump.

2. The device in accordance with claim 1, wherein the control is adapted to carry out at least one of the steps in an inflow phase of a peritoneal dialysis treatment and/or in an outflow phase of a peritoneal dialysis treatment.

3. A device in accordance with claim 1, wherein the control is adapted only to open a valve fluidically connecting the first pump to the second pump when the second pump has completely ended a last pump stroke and/or is inactive.

4. The device in accordance with claim 1, wherein the first and the second pump each interact with a pump chamber formed in a disposable to convey fluid.

5. The device in accordance with claim 1, wherein use is made of a pressure profile from a database associated with the unit properties, with a valve switching, as part of the calculatory compensation in order thus to identify and compensate the portion of the result of a pressure measurement based on the unit properties during a switching time of at least one of the valves.

6. The device in accordance with claim 1, wherein the control is adapted to make use of the optimization algorithm to determine a point in time or to calculate a point in time in advance to which the valve switching point in time of the second pump is postponed, with the optimization algorithm taking account of at least one of the following parameters at a given point in time: Flow rate of the first and/or of the second pump; last conveyed pump chamber volume of the first and/or second pump(s); volume present in the patient and type of a treatment carried out.

7. The device in accordance with claim 6, wherein the optimization algorithm is adapted to minimize a time delay between a valve switching point in time of the second pump to start a pump stroke of the second pump and a valve switching point in time of the first pump on the ending of a pump stroke of the first pump.

8. The device in accordance with claim 1, wherein step c) includes a postponement of the valve switching point in time of the second pump by a fixed delay period starting from a valve switching point in time of the first pump such that the valve switching point in time of the second pump does not coincide with the valve switching point in time of the first pump.

9. The device in accordance with claim 1, wherein step d) includes a postponement of the valve switching point in time of the second pump by a variable delay period, starting from a valve switching point in time of the first point such that the valve switching point in time of the second pump does not coincide with the valve switching point in time of the first pump.

10. A method of generating a continuous volume flow of dialyzate by means of a device for extracorporeal blood treatment having first and second discontinuous pumps that are switchable by means of at least two valves, wherein the method comprises at least one of the following steps: a) suspending at least one pressure measurement during a switching time of at least one of the valves; b) calculatory compensation of a portion of a result of a pressure measurement based on unit properties during a switching time of at least one of the valves; c) postponing a valve switching point in time of the second pump so that the valve switching point in time of the second pump does not coincide with a valve switching point in time of the first pump; and d) adaptive postponement of a valve switching point in time of the second pump so that said valve switching point in time does not coincide with a valve switching point in time of the first pump, wherein use is made of an optimization algorithm for determining a point in time to which the valve switching point in time of the second pump is postponed.

11. The method accordance with claim 10, wherein at least one of the steps is carried out in an inflow phase of a peritoneal dialysis treatment and/or in an outflow phase of a peritoneal dialysis treatment.

12. The method in accordance with claim 10, wherein a valve fluidically connecting the first pump to the second pump is only opened when the second pump has completely ended a last pump stroke and/or is inactive.

13. The method in accordance with claim 10, wherein use is made of a pressure profile from a database associated with the unit properties, with a valve switching, as part of the calculatory compensation in order thus to identify and compensate the portion of the result of a pressure measurement based on the unit properties during a switching time of at least one of the valves.

14. The method in accordance with claim 10, wherein use is made of the optimization algorithm to determine a point in time or to calculate a point in time in advance to which the valve switching point in time of the second pump is postponed, with the optimization algorithm taking account of at least one of the following parameters at a given point in time: Flow rate of the first and/or of the second pump, flow rate of the pump of the first and second pumps that is active at the point in time; last conveyed pump chamber volume of the first and/or second pump(s), the flow rate of the pump of the first and second pumps that is active at the point in time; volume present in the patient and type of a treatment carried out.

15. The method accordance with claim 14, wherein the optimization algorithm is adapted to minimize a time delay between a valve switching point in time of the second pump to start a pump stroke of the second pump and a valve switching point in time of the first pump on the ending of a pump stroke of the first pump.

16. The method in accordance with claim 10, wherein step c) includes a postponement of the valve switching point in time of the second pump by a fixed delay period starting from a valve switching point in time of the first pump such that the valve switching point in time of the second pump does not coincide with the valve switching point in time of the first pump or deviates therefrom in time.

17. The method in accordance with claim 10, wherein step d) includes a postponement of the valve switching point in time of the second pump by a variable delay period, individually determined by the optimization algorithm for every valve switching procedure, starting from a valve switching point in time of the first point such that the valve switching point in time of the second pump does not coincide with the valve switching point in time of the first pump.

18. The device for dialysis treatment in accordance with claim 1, wherein the device is for peritoneal dialysis, and wherein use is made by the control of an optimization algorithm for determining a point in time to which the valve switching point in time of the second pump is postponed.

19. The device in accordance with claim 1, wherein the first and the second pump each interact with a pump chamber formed in a disposable to convey fluid and the at least two valves are each components of the disposable.

20. The device in accordance with claim 1, wherein the control is adapted to make use of the optimization algorithm to determine a point in time or to calculate a point in time in advance to which the valve switching point in time of the second pump is postponed, with the optimization algorithm taking account of at least one of the following parameters at a given point in time: Flow rate of the first and/or of the second pump, flow rate of the pump of the first and second pumps that is active at the point in time; last conveyed pump chamber volume of the first and/or second pump(s), the flow rate of the pump of the first and second pumps that is active at the point in time; volume present in the patient and type of a treatment carried out.
